(19) United States
(12) Patent Application Publication
(10) Pub. No.: US 2022/0076584 A1
(43) Pub. Date: Mar. 10, 2022

(54) SYSTEM AND METHOD FOR PERSONALIZED HEALTHCARE STAFF TRAINING

(71) Applicant: KONINKLIJKE PHILIPS N.V., EINDHOVEN (NL)

(72) Inventors: Jin LIU, MALDEN, MA (US); Puran Adesh BHAGAT, ANDOVER, MA (US)

(21) Appl. No.: 17/469,990

(22) Filed: Sep. 9, 2021

Related U.S. Application Data
(60) Provisional application No. 63/075,855, filed on Sep. 9, 2020.

(51) Int. Cl. G09B 5/02; G16H 40/20

(52) U.S. Cl. CPC G09B 5/02; G16H 40/20

(57) ABSTRACT

A method (100) of providing training content to medical professionals includes: retrieving (i) historical data related to staff performance of staff members of a medical facility and (ii) training data about past training received by the staff members; computing key performance indicators (KPIs) for the staff members using the retrieved historical data; estimating, using the retrieved historical data, a first improvement score for the KPIs that is predicted to result from consumption of a corresponding training content unit (30); estimating a second improvement score for the KPIs based on information related to requirements or goals for training the staff members; combining the first and second improvement scores to generate a combined score; and generating one or more recommended training content units based on the combined score and the retrieved training data.

This application claims the benefit of U.S. Provisional Application No. 63/075,855 filed on Sep. 9, 2020. This application is hereby incorporated by reference herein.

### FIELD

The following relates generally to personalized training arts, healthcare staff training arts, health care staff training recommendation arts, data-driven model arts, hybrid model arts, performance improvement prediction arts, and related arts.

### BACKGROUND

Healthcare is an active area with continuous developments in clinical knowledge and frequent new technology and new software releases. Therefore, healthcare staff members need proper and timely training on how to perform properly and efficiently in this rapidly changing environment to get optimal operational performance.

Meanwhile, under the pressure of reduced reimbursement and the shift to value-based healthcare, healthcare managers are under the pressure of providing better value with less cost. With associated cost of training and unproductive time of staff members during training, how to balance the benefit and cost of training is a challenge to healthcare managers.

In addition, different staff members may need personalized training due to differences in job duties, a priori knowledge, training retention, and other factors, making it difficult to find the optimal training time/content for all staff members. For example, a few professionals can make a mistake by recording the wrong procedure type, while other medical professionals often forget to record patient emergency status. It would be beneficial to identify who made frequent mistake (i.e., underperform) on which task to train the specific staff members on specific issue to gain the optimal improvement.

For example, in regards to medical imaging, with the continuous release of new features of automatic image reading capability, some imaging technicians such as sonographers or cardiologists could be unaware of them without timely training, or otherwise forget these features after a while.

The following discloses certain improvements to overcome these problems and others.

### SUMMARY

In one aspect, a non-transitory computer readable medium stores instructions executable by at least one electronic processor to perform a method of providing training content to medical professionals. The method includes: retrieving (i) historical data related to staff performance of staff members of a medical facility and (ii) training data about past training received by the staff members; computing key performance indicators (KPIs) for the staff members using the retrieved historical data; estimating, using the retrieved historical data, a first improvement score for the KPIs that is predicted to result from consumption of a corresponding training content unit; estimating a second improvement score for the KPIs based on information related to requirements or goals for training the staff members; combining the first and second improvement scores to generate a combined score; and generating one or more recommended training content units based on the combined score and the retrieved training data.

In another aspect, a non-transitory computer readable medium stores instructions executable by at least one electronic processor to perform a method of providing training content to medical professionals. The method includes: retrieving (i) historical data related to staff performance of staff members of a medical facility and (ii) training data about past training received by the staff members; computing KPIs for the staff members using the retrieved historical data; estimating, using the retrieved historical data, a first improvement score for the KPIs that is predicted to result from consumption of a corresponding training content unit, the estimating including inputting the retrieved data into a data driven model; estimating a second improvement score for the KPIs based on information related to requirements or goals for training the staff members; combining the first and second improvement scores to generate a combined score as a weighted combination of the first improvement score and the second improvement score; and generating one or more recommended training content units based on the combined score and the retrieved training data.

In another aspect, a non-transitory computer readable medium stores instructions executable by at least one electronic processor to perform a method of providing training content to medical professionals. The method includes: retrieving (i) historical data related to staff performance of staff members of a medical facility and (ii) training data about past training received by the staff members; computing KPIs for the staff members using the retrieved historical data; estimating, using the retrieved historical data, a first improvement score for the KPIs that is predicted to result from consumption of a corresponding training content unit; estimating a second improvement score for the KPIs based on information related to requirements or goals for training the staff members; combining the first and second improvement scores to generate a combined score; generating one or more recommended training content units based on the combined score and the retrieved training data; providing a manager user interface (UI) at an electronic device operable by a manager of the medical facility, the UI including fields to display the KPIs and a list of the recommended content units with corresponding combined scores; and displaying a list of the recommended content units for a staff member on an electronic device operable by the staff member.

One advantage resides in tailoring training units to individual medical professionals.

Another advantage resides in improving productivity of medical professionals after receiving tailored training.

Another advantage resides in monitoring individual medical professionals' behaviors to recommend appropriate training units.

Another advantage resides in providing training units to be completed when the corresponding medical professional is available.

Another advantage resides in providing training content recommendations on the basis of both historical data on staff members' performance and past training, and also predicted improvement in key performance indicators (KPIs) due to consuming the training content.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for providing training content to medical professionals in accordance with the present disclosure.

FIG. 2 shows modules implemented by the system of FIG. 1.

FIG. 3 shows exemplary flow chart operations of the system of FIG. 1.

FIGS. 4 and 5 show example graphical user interfaces generated by the system of FIG. 1.

### DETAILED DESCRIPTION

The following relates to targeted providing of healthcare training. When a new product or software upgrade is released, training is usually provided. However, this can be insufficient if the hospital is experiencing staff turnover, or if the training should be repeated at intervals either to improve retention or to meet regulatory requirements or goals.

The disclosed system collects information about staff performance, such as for specific key performance indicators (KPIs), and staff training. Such information is fed into a data driven model that estimates an improvement score for each KPI that is predicted to result from consumption of a particular training unit. The modeling includes data driven models for each (KPI, training unit) pair, and the model can be applied for each staff member based on inputs including performance and training records of that staff member.

While such a data driven modeling approach is useful, it may be limited if the source data is deficient (for example, a newly hired employee, or deficiencies in the hospital's record-keeping). Accordingly, a second, rules-based model can be employed. This model can capture information such as regulatory or hospital requirements or goals for training (e.g., a particular training unit must be repeated every three years). The rules-based model can also be useful where data for using the data-driven model is deficient.

To combine the scores for a (KPI, training unit) pair generated by the data-driven model and the rules-based model, a hybrid model employs a weighted combination of the data-driven and rules-based scores. The relative weights for the two scores may be set based on a metric of quality of the data supplied to the data-driven model, the strength of the rules (e.g., a mandatory rule may result in the rules-based score being assigned a very high weight), or may be set manually by an administrator.

A computer-implemented performance monitoring/training recommendation module takes as input staff members' KPIs and training data, and the scores generated by the hybrid model, and generates training recommendations. A manager's user interface (UI) provides information such as the historical KPIs for specific staff members and predictions on potential improvement due to a training unit (determined by the scores of the hybrid model) and costs for the training unit. The manager's UI allows the manager to select specific KPIs to display.

A staff UI is also provided, via which a staff member can update his or her training history if information is not captured by the data mining. The staff UI also provides the staff member with a record of training performed and training scheduled or overdue.

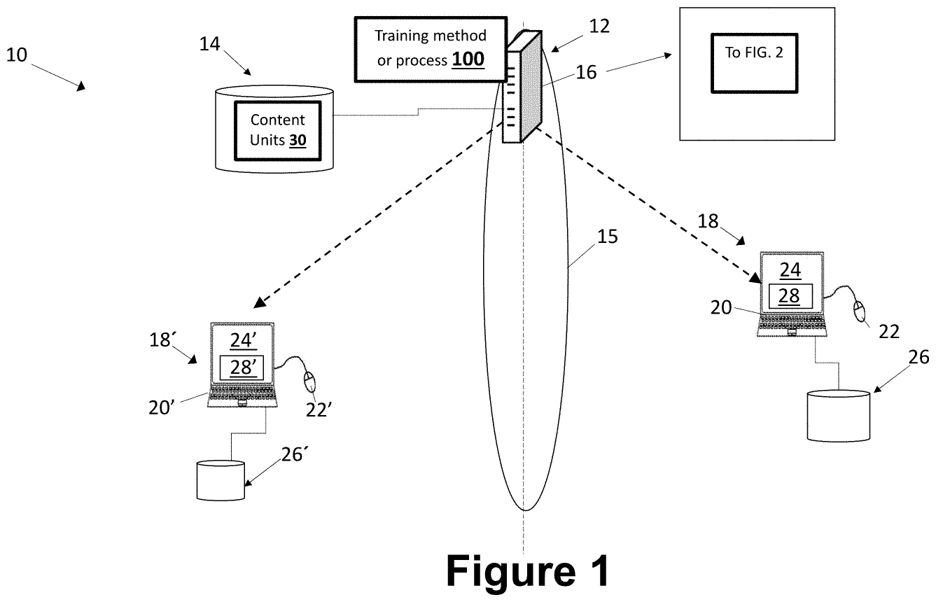

With reference to FIG. 1, an apparatus or system 10 for providing training content to medical professionals is shown. As shown in FIG. 1, the apparatus 10 comprises a server computer 12 having a database or non-transitory computer readable medium 14 storing instructions executable by at least one electronic processor 16 of the server computer. The server computer 12 is in communication with a first remote electronic processing device 18 operable by a manager of a team of medical professionals to provide input related to recommended training content for the members of the medical professional team. The manager electronic processing device 18 can be any suitable electronic processing device, such as a workstation computer (or more generally a computer), a smartphone, a tablet, and so forth. Additionally or alternatively, the manager electronic processing device 18 can be embodied as a server computer or a plurality of server computers, e.g. interconnected to form a server cluster, cloud computing resource, or so forth. FIG. 1 shows the manager electronic processing device 18 as a workstation computer. The manager workstation 18 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the manager display device 24.

The server computer 12 is also in communication with a second remote electronic processing device 18′ operable by one or more members of the manager's team. The staff electronic processing device 18′ can be any suitable electronic processing device, such as a workstation computer (or more generally a computer), a smartphone, a tablet, and so forth. For illustrative purposes, in FIG. 1, only one staff electronic processing device 18′ is shown (e.g., a computer at a hospital where the medical professionals work, and can be accessed by multiple team members); however, the server computer 12 may be in communication with several staff electronic processing devices 18′ (e.g., each team member's cellphone, tablet, or personal computer). As a further contemplated variant, the manager electronic processing device 18 could also serve as one of the staff electronic processing devices 18′. As shown in FIG. 1, the staff electronic processing device 18′ also includes similar components as the manager workstation 18. Except as otherwise indicated herein, features of the staff workstation 18′ similar to those of the manager workstation 18 have a common reference number followed by a “prime” symbol, and the description of the components of the staff electronic processing device 18′ will not be repeated. In particular, the staff workstation 18′ is configured to display a GUI 28′ on a display device or controller display 24′ that presents information pertaining to the training content.

The server computer 12 can be in communication with the manager workstation 18, and the staff workstation 18′ via a communication link 15 which typically comprises the Internet augmented by local area networks at the manager and staff ends for electronic data communications.

The database 14 of the server computer 12 stores records related to job performance of the medical professionals (e.g., task lists, reviews, performance evaluations, experience records, and so forth) on the manager's team. Data related to training of new or existing team members is also stored in the database 14. In addition, a plurality of training content units 30 for training the staff members are stored in the database 14. One or more modules are implemented by the electronic processor 16 of the server computer 12 to push recommended content units 30 to the staff electronic processing device (s) 18′.

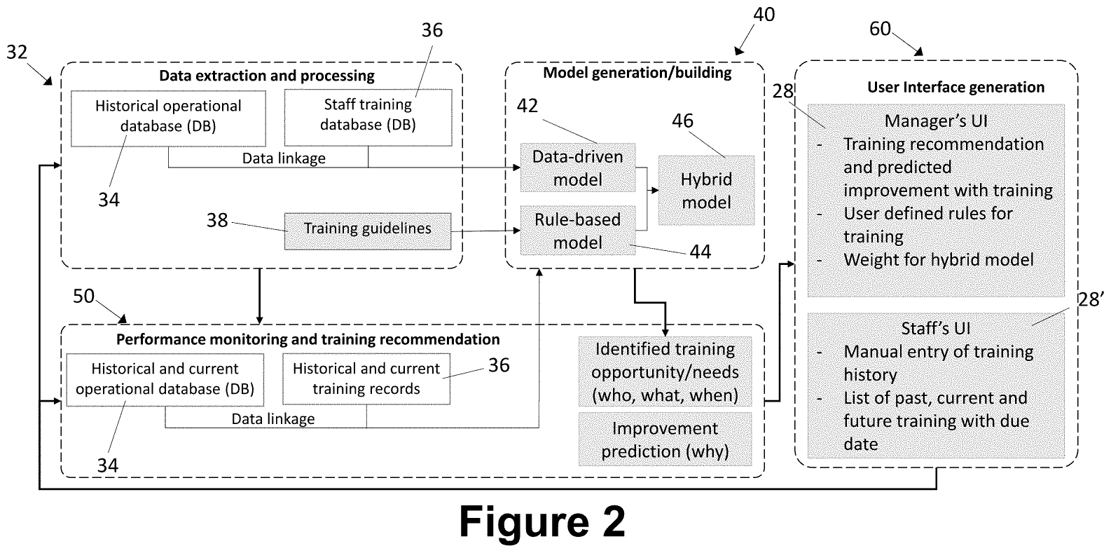

With further reference to FIG. 2 and continuing reference to FIG. 1, FIG. 2 shows examples of the modules executable by the electronic processor 16 of the server computer 12. An extraction module 32 is configured to extract and process operational information, training activities and guidelines from the records and data stored in the database 14. In addition, the extraction module 32 is programmed to extract historical and current operational information from multiple healthcare informatics systems 34 (e.g., an Electronic Medical Records (EMR) database, a Cardiology Information System (CIS database, and so forth), which are not shown in FIG. 2. The extracted data has detailed elements (e.g. the operating staff member, what procedure at which time, what machine/software feature was used and what was recorded at each time point, and so forth). The performed extraction and processing depend on the format of the database content. For databases containing structured information, such as a relational database, SQL queries may be employed. For databases containing free text information (e.g., free form text documents), keyword searching and/or natural language processing (NLP) may be employed. If a database is accessed by a standardized transfer protocol such as Health Level 7 (HL7), then HL7 compliant database access can be employed. These are merely non-limiting illustrative examples. With the extracted information, KPIs (e.g. turnaround time, on-time start, productivity) can be calculated based on predefined definition. The manager optionally has the ability to modify the definitions of the KPIs via the manager workstation 18.

In addition, the extraction module 32 is also configured to extract historical and current training activities of the staff members. This data can be retrieved from an electronic training documentation database 36. Data processing (e.g. data cleaning, key word matching, NLP or manual entry) could be used to make this dataset structured with elements of the staff members (i.e., staff ID or name), time of training, and content of training.

The staff data and the operational data extracted by the extraction module 32 can be linked by the name of the staff member performing the operation, by a shared ID, or by a linkage file storing paired staff IDs. A quality check operation can be implemented in case of common or unmatched names or IDs. The extraction module 32 is also configured to retrieve or extract training guidelines 38 stored in the database 14 using an automatic method (e.g., NLP) or manual entry via the manager using the manager workstation 18 to build rules of training conditions and content of training if condition is met.

A building or generation module 40 is configured to build a training recommendation model. The building module 40 is programmed to use a data-driven model 42 and a rules-based model 44 to generate a hybrid training recommendation model 46. The hybrid model 46 is used to find training contents unit 30, content for the training content units, and the effectiveness of training content units.

The historical staff performance data and the historical training activities data extracted by the extraction module 32 are input to the data-driven model 42 at different time intervals. The data-driven model 42 can be generated by the building module 40 using, for example, machine-learning (ML) algorithms (e.g., classification algorithms, regression algorithms, and so forth) and/or deep-learning (DL) algorithms (such as a recurrent neural network (RNN)) to predict training needed (i.e., classification) and potential improvement (i.e., regression). Features to build the data-driven model 42 could be selected using, for example, a best subset method or a Lasso regularization operation. A scaled score, (s₁), (e.g. 0-100) can be a weighted average based on the potential improvement (s₁₁) and the confidence score (s₁₂) calculated, along with their respective weights (w₁₁) and (w₁₂) according to Equation 1:

s₁=w₁₁·s₁₁+w₁₂·s₁₂, where w₁₁+w₁₂=1 (1)

The rules-based model 44 is used to generate rules as to whether training is needed or not (i.e., a yes/no output) and a scaled score, (s₂), is calculated to reflect the percentage difference with a cut-off threshold.

The data-driven model 42 and the rules-based model 44 are combined by the building module 40 to generate the hybrid model 46 with a weighted average score. Via the manager workstation 18, the manager has the ability to assign weights (w₁ and w₂) to the hybrid model 46 with informed knowledge of automated weight suggestion from a training data quality assessment (e.g. a lower weight of (w₁) with limited training data for a data-driven method), according to Equation 2:

s=w₁·s₁+w₂·s₂, where w₁+w₂=1 (2)

The data-driven model 42 predicts training that is normally performed at specific time intervals with varying KPIs based on previous practice. The rules-based model 44 predicts training that should be performed based on predefined rules. If the extracted historical data is of good quality (e.g. a large amount of data from the healthcare organization with good training practice), more weight or trust can be put to data-driven model 42; while if the historical data is of poor quality (e.g. limited data from small regional hospital), more weight or trust can be put into the rule-based model 44. Optionally, default weights may be assigned automatically based on information such as the amount of data input to the data-driven model, quality metrics of that data, or other available information, and the manager has the option of changing the default weights.

A recommendation module 50 is configured to monitor staff performance and suggest needed training (i.e., specific content units selected from the content units 30). To do so, the recommendation module 50 is configured to monitor current operational performance of the staff members in real-time, and suggest who need training and which content units 30 should be recommended. Using the data extracted by the extraction module 32 and the hybrid model 46 generated by the building module 40, the recommendation model is configured to extract and monitor data related to the performance of the staff members in their operational and/or clinical tasks. When the monitored performance data falls below a predetermined threshold, or if a training rule is satisfied, the recommendation module 50 is configured to recommend one or more corresponding training content units 30. The recommended content units 30 are tailored for each staff member based on that staff member's performance in different operations or topics. A potential improvement in a staff member's performance, after consuming the recommended content unit(s) 30 can be estimated or predicted based on the data-driven model 42. This performance forecasting can also be used to predict future content units 30 to be recommended. Such methods for performance forecasting can include, for example, time series forecasting, multivariate regression, recurrent neural networks, and/or other combined methods. In this way, future recommended content units 30 can be predicted ahead of time to plan activities earlier for the manager and staff members.

A user interface (UI) generation module 60 is configured to generate respective UIs 28 and 28′ for the manager and the staff members on the respective electronic processing devices 18 and 18′. By way of non-limiting illustrative example, on the GUI 28, a field is provided which shows performance improvement if suggested training is adopted (i.e., the recommended content units 30 are consumed by the corresponding staff members). Based on the estimated performance improvement of suggested training, the manager can better decide based on the return on investment (ROI). In addition, on the GUI 28, rule-based training guidelines can be modified (e.g., adding new rules, deleting rules, changing rules, and so forth) based on the manager's preference.

On the staff GUI 28′, the respective staff members can manually (e.g., via the at least one user input device 22′) enter their training history if missed by the system. In addition, staff members can visualize which content units 30 have been consumed, and which content units are scheduled or overdue.

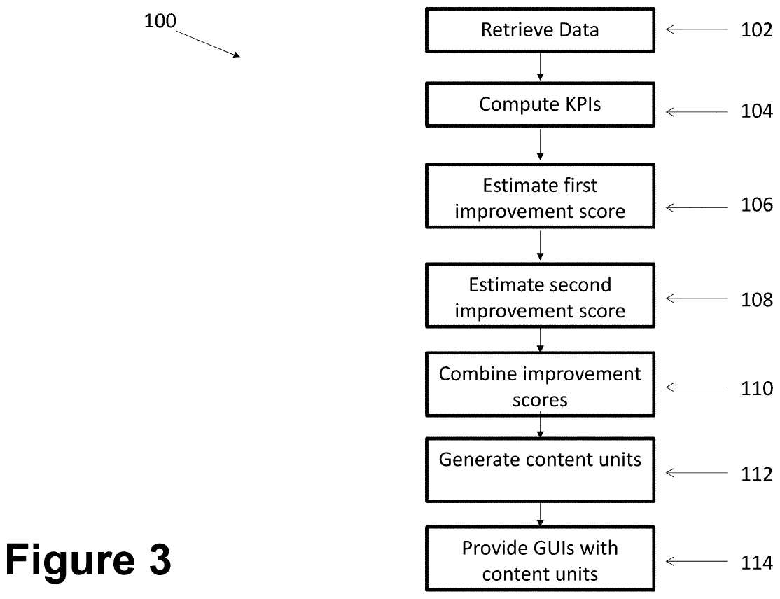

With reference to FIG. 3, and with continuing reference to FIGS. 1 and 2, the at least one electronic processor 16 of the server computer 12 is configured as described above to perform a method or process 100 for providing training content to medical. The non-transitory storage medium 14 stores instructions which are readable and executable by the at least one electronic processor 16 to perform disclosed operations including performing the method or process 100. In some examples, the method 100 may be performed at least in part by cloud processing.

At an operation 102, data is retrieved from one or more databases. The retrieved data can include (i) historical data related to staff performance of the staff members of the medical facility and (ii) training data about past training received by the staff members. The retrieving operation 102 can be performed by the extraction module 32.

At an operation 104, one or more KPIs are computed for the staff members using the retrieved historical data. The computing operation 104 can be performed by the extraction module 32.

At an operation 106, a first improvement score is estimated for the KPIs using the retrieved historical data. The first improvement score is predicted to result from consumption of a corresponding training content unit 30. The first estimating operation 106 can be performed by the building module 40.

In some examples, the first estimating operation 106 includes inputting the retrieved data (from the operation 102) into the data-driven model 42. The first improvement score comprises an output of the data-driven model 42. In other embodiments, the data-driven model 42 comprises a plurality of data-driven models corresponding to the number of KPIs calculated at the calculating operation 104. The retrieved data from the retrieving operation 102 is input into each data-driven model 42 to generated KPI-content unit pairs. The first improvement score is based on the KPI-content unit pairs. A first improvement score can be determined for each staff member based on data related to performance and training records for each staff member.

At an operation 108, a second improvement score is estimated for the KPIs based on information related to requirements or goals for training the staff members. The second estimating operation 108 can be performed by the building module 40. (It is noted that the order of the operations 106 and 108 can be reversed, or if sufficient computing capacity is available then the operations 106 and 108 can be performed concurrently).

At an operation 110, the first and second improvement scores are combined to generate a combined score. In some embodiments, the first and second improvement scores are combined for each KPI-content unit pair. In other embodiments, the combining operation 110 includes generating the combined score as a weighted combination of the first improvement score and the second improvement score. In one example, the weights for the weighted combination can include a metric of quality of the retrieved historical data. In another example, the weights for the weighted combination can include a strength setting of the requirements or goals for training the staff members. In a further example, the weights for the weighted combination can include a manual entry of the weights by the manager via the at least one user input device 22.

At an operation 112, one or more recommended training content units 30 are generated based on the combined score and the retrieved training data.

In some embodiments, the computing operation 104, the estimating operations 106 and 108, the combining operation 110, and the generating operation 112, can be performed for each individual staff member to recommend training content units 30 for each individual staff member. In this embodiment, the generating operation 112 is further based on a cost of delivering the training content units to the individual staff members.

At an operation 114, a GUI 28, 28′ is provided at a corresponding electronic processing device 18, 18′. In some embodiments, the GUI 28 includes fields to display the KPIs and a list of the recommended content units 30 with corresponding combined scores. In other embodiments, the GUI 28′ includes a list of the recommended content units 30 for the staff members.

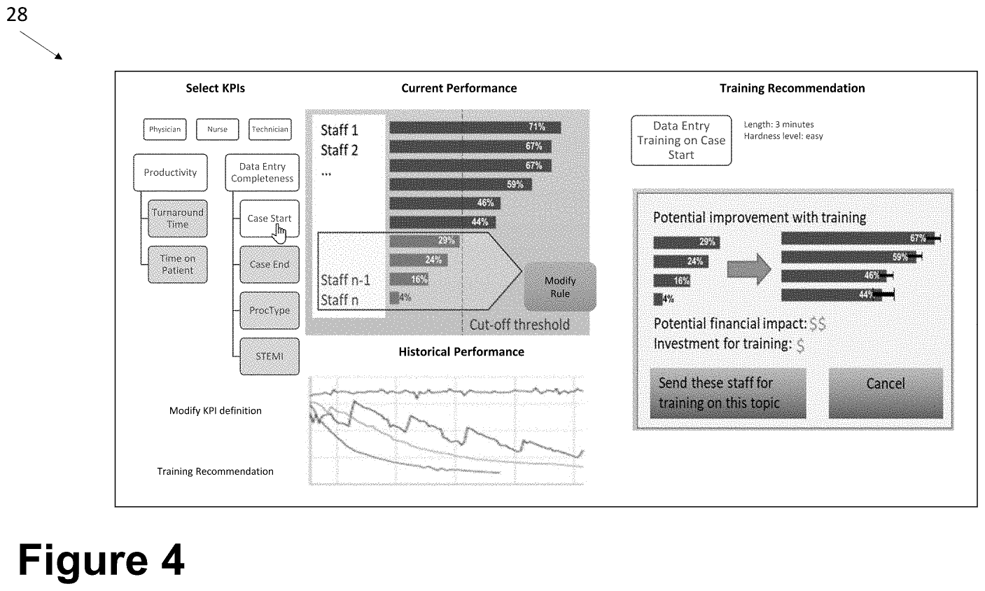

FIG. 4 shows an example of the GUI 28 displayed on the manager workstation 18. As shown in FIG. 4, the GUI 28 can include fields related the selection of KPIs, refining the definition of the KPIs, a current performance of the staff members, recommended content units 30, potential improvement with training, and so forth.

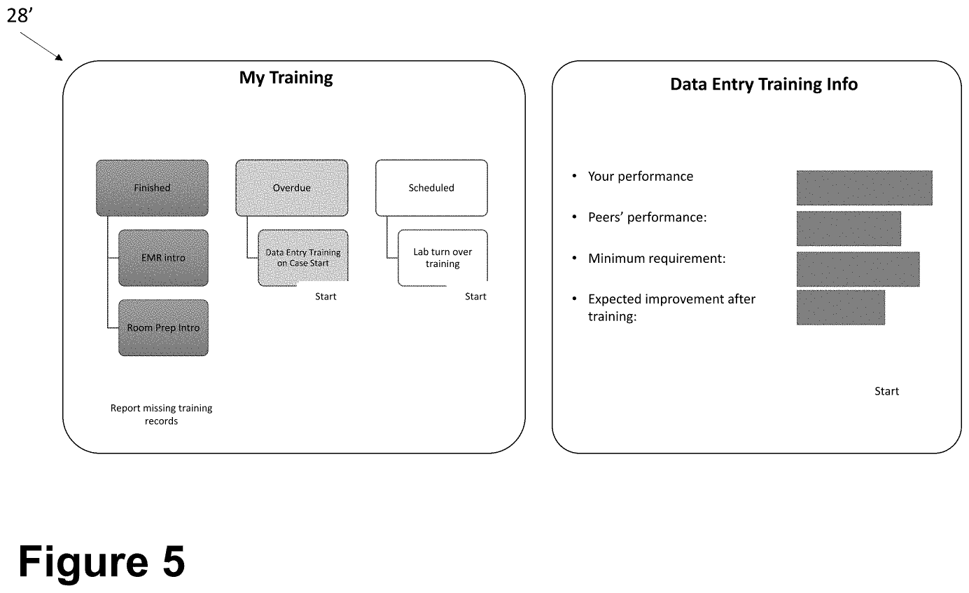

FIG. 5 shows an example of the GUI 28′ displayed on the staff workstation 18. As shown in FIG. 4, the GUI 28′ can include fields related the training of individual staff members, data entry training information, and so forth.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of providing training content to medical professionals, the method comprising:
retrieving (i) historical data related to staff performance of staff members of a medical facility and (ii) training data about past training received by the staff members;
computing key performance indicators (KPIs) for the staff members using the retrieved historical data;
estimating, using the retrieved historical data, a first improvement score for the KPIs that is predicted to result from consumption of a corresponding training content unit;
estimating a second improvement score for the KPIs based on information related to requirements or goals for training the staff members;
combining the first and second improvement scores to generate a combined score; and
generating one or more recommended training content units based on the combined score and the retrieved training data.

2. The non-transitory computer readable medium of claim 1, wherein the combining includes:
generating the combined score as a weighted combination of the first improvement score and the second improvement score.

3. The non-transitory computer readable medium of claim 2, wherein the weights for the weighted combination includes a metric of quality of the retrieved historical data.

4. The non-transitory computer readable medium of claim 2, wherein the weights for the weighted combination includes a strength setting of the requirements or goals for training the staff members.

5. The non-transitory computer readable medium of claim 2, wherein the weights for the weighted combination includes a manual entry of the weights via at least one user input device.

6. The non-transitory computer readable medium of claim 1, wherein the estimating of the first improvement score includes:
inputting the retrieved data into a data driven model; and
determining the first improvement score as the output of the data driven model.

7. The non-transitory computer readable medium of claim 6, wherein the determining includes:
inputting the retrieved data into a plurality of different data driven models for respective KPIs to generate KPI-content unit pairs; and
determining the first improvement score based on the KPI-content unit pairs.

8. The non-transitory computer readable medium of claim 7, wherein the determining includes determining a first improvement score for each staff member based on data related to performance and training records for each staff member.

9. The non-transitory computer readable medium of claim 7, wherein the combining of the first improvement score and the second improvement score includes:
combining the first and second improvement scores for each KPI-content unit pairs.

10. The non-transitory computer readable medium of claim 1, wherein the method further includes:
performing the computing of the KPIs, the estimating of the first and second improvement scores for the KPIs, the combining, and the generating to provide recommended training content units (30) for individual staff members.

11. The non-transitory computer readable medium of claim 10, wherein the generating of the training content units for individual staff members is further based on a cost of delivering the training content units to the individual staff members.

12. The non-transitory computer readable medium of claim 1, wherein the method further includes:
providing a manager user interface (UI) at an electronic device operable by a manager of the medical facility, the UI including fields to display the KPIs and a list of the recommended content units with corresponding combined scores.

13. The non-transitory computer readable medium of claim 1, wherein the method further includes:
displaying a list of the recommended content units for a staff member on an electronic device operable by the staff member.

14. A non-transitory computer readable medium storing instructions executable by at least one electronic processor to perform a method of providing training content to medical professionals, the method comprising:
retrieving (i) historical data related to staff performance of staff members of a medical facility and (ii) training data about past training received by the staff members;
computing key performance indicators (KPIs) for the staff members using the retrieved historical data;
estimating, using the retrieved historical data, a first improvement score for the KPIs that is predicted to result from consumption of a corresponding training content unit, the estimating including inputting the retrieved data into a data driven model;
estimating a second improvement score for the KPIs based on information related to requirements or goals for training the staff members;
combining the first and second improvement scores to generate a combined score as a weighted combination of the first improvement score and the second improvement score; and
generating one or more recommended training content units based on the combined score and the retrieved training data.

15. The non-transitory computer readable medium of claim 14, wherein the weights for the weighted combination includes at least one of:
a metric of quality of the retrieved historical data;
a strength setting of the requirements or goals for training the staff members; and
a manual entry of the weights via at least one user input device.

16. The non-transitory computer readable medium of claim 14, wherein the determining of the first improvement score includes:
inputting the retrieved data into a plurality of different data driven models for respective KPIs to generate KPI-content unit pairs; and
determining the first improvement score based on the KPI-content unit pairs for each staff member based on data related to performance and training records for each staff member.

17. The non-transitory computer readable medium of claim 16, wherein the combining of the first improvement score and the second improvement score includes:
combining the first and second improvement scores for each KPI-content unit pairs.

18. The non-transitory computer readable medium of claim 14, wherein the method further includes:
providing a manager user interface (UI) at an electronic device operable by a manager of the medical facility, the UI including fields to display the KPIs and a list of the recommended content units with corresponding combined scores.

19. The non-transitory computer readable medium of claim 14, wherein the method further includes:
displaying a list of the recommended content units for a staff member on an electronic device operable by the staff member.

20. A non-transitory computer readable medium storing instructions executable by at least one electronic processor to perform a method of providing training content to medical professionals, the method comprising:
retrieving (i) historical data related to staff performance of staff members of a medical facility and (ii) training data about past training received by the staff members;
computing key performance indicators (KPIs) for the staff members using the retrieved historical data;
estimating, using the retrieved historical data, a first improvement score for the KPIs that is predicted to result from consumption of a corresponding training content unit;
estimating a second improvement score for the KPIs based on information related to requirements or goals for training the staff members;
combining the first and second improvement scores to generate a combined score;
generating one or more recommended training content units based on the combined score and the retrieved training data;
providing a manager user interface (UI) at an electronic device operable by a manager of the medical facility, the UI including fields to display the KPIs and a list of the recommended content units with corresponding combined scores; and
displaying a list of the recommended content units for a staff member on an electronic device operable by the staff member.
